(19) United States
(12) Patent Application Publication
(10) Pub. No.: US 2019/0142358 A1
(43) Pub. Date: May 16, 2019

(54) Method And System For Dose-Less Attenuation Correction For PET And SPECT

(71) Applicant: Siemens Medical Solutions USA, Inc., Malvern, PA (US)

(72) Inventors: Terrence Chen, Princeton, NJ (US); Vivek Kumar Singh, Princeton, NJ (US); Klaus J. Kirchberg, Plainsboro, NJ (US); Vladimir Y. Panin, Knoxville, TN (US); Dorin Comaniciu, Princeton Junction, NJ (US)

(21) Appl. No.: 15/810,818

(22) Filed: Nov. 13, 2017

(51) Int. Cl. A61B 6/00; G06T 11/00; A61B 6/03; A61B 6/04; A61B 5/00; G01T 1/29

(52) U.S. Cl. CPC A61B 6/5258; G06T 11/005; A61B 6/032; A61B 6/037; A61B 6/0407; G01T 1/2985; A61B 5/0077; A61B 6/5205; A61B 5/7278; A61B 5/0035; A61B 6/5235

(57) ABSTRACT

A method for generating a nuclear image includes obtaining, via a camera, a surface image of a patient. A synthetic computed-tomography (CT) image of the patient is generated based on the surface image. First time-of-flight (TOF) data for the patient is obtained via a nuclear imaging modality. Attenuation correction is applied to the first TOF data. The synthetic image is applied as a density map during the attenuation correction. A nuclear image is generated from the attenuation corrected first TOF data.

### FIELD

Aspects of the present disclosure relate in general to nuclear imaging systems, and more particularly to attenuation correction for nuclear imaging systems.

### BACKGROUND

Time-of-flight (TOF) nuclear imaging, such as TOF positron emission tomography (PET), is used to construct two-dimensional and/or three-dimensional images of structures within a patient. TOF PET (and other TOF nuclear imaging) detects coincidence events representing near simultaneous detection of annihilation photon pairs using a pair of detectors. The TOF PET system determines the difference in time between the detection of the two photons (e.g., the time of flight) and localizes the point of origin of the annihilation event that occurred between the two detectors.

Attenuation correction is applied to nuclear imaging, such as PET imaging, to correct for artifacts that may occur during a scan, including prominent activity at body surface because of a lack of attenuation modeling at surfaces, distorted appearance of areas of intense activity, and diffuse, relatively increased activity in tissues of low attenuation. Current systems use a computerized-tomography (CT) scan to build an attenuation map of density differences in the whole body, which can be used to correct the absorption of the photons. The use of a CT scan increase cost and require additional doses to a patient.

### SUMMARY

A method for generating a nuclear image is disclosed. The method comprising: obtaining, via a camera, a surface image of a patient; generating a synthetic computed-tomography (CT) image of the patient based on the surface image; obtaining, via a nuclear imaging modality, first time-of-flight (TOF) data of the patient; applying attenuation correction to the first TOF data, wherein the synthetic CT image is applied as a density map during the attenuation correction; and generating a nuclear image from the attenuation corrected first TOF data.

A system for generating a nuclear image is also disclosed. The system comprising: a gantry sized and configured to receive a patient; a scanner including a first imaging modality configured to detect a first plurality of photon events, the first imaging modality comprising a plurality of detectors; a camera configured to obtain a surface image of the patient; and a processor configured to receive a signal indicative of the first plurality of photon events and the surface image. The processor is configured to generate a synthetic computed-tomography (CT) image of the patient based on the surface image, apply attenuation correction to the first plurality of photon events, wherein the synthetic image is applied as a density map during the attenuation correction, and generate a nuclear image from the attenuation corrected plurality of photon events.

A non-transitory computer-readable medium encoded with computer executable instructions is also disclosed. The computer executable instructions, when executed by a computer in a system for generating a nuclear image, cause the system for generating a nuclear image to execute the steps of: obtaining a surface image of a patient; generating a synthetic computed-tomography (CT) image of the patient based on the surface image; obtaining first time-of-flight (TOF) data of the patient; applying attenuation correction to the first TOF data, wherein the synthetic CT image is applied as a density map during the attenuation correction; and generating a nuclear image from the attenuation corrected first TOF data.

### BRIEF DESCRIPTION OF THE DRAWINGS

The following will be apparent from elements of the figures, which are provided for illustrative purposes and are not necessarily drawn to scale.

FIG. 1 illustrates a nuclear imaging system, in accordance with some embodiments.

FIG. 2 illustrates a method of generating a combined nuclear image using a synthetic CT scan, in accordance with some embodiments.

FIG. 3 illustrates various steps of the method of FIG. 2, in accordance with some embodiments.

FIG. 4 illustrates a method of generating a combined nuclear image having an iterative artifact reduction process, in accordance with some embodiments.

FIG. 5 illustrates various steps of the method of FIG. 4, in accordance with some embodiments.

FIG. 6 illustrates a method of generating a combined nuclear image using a synthetic CT scan and an uncorrected PET scan, in accordance with some embodiments.

FIG. 7 illustrates various steps of the method of FIG. 6, in accordance with some embodiments.

FIG. 8 illustrates a method of generating a nuclear image using a synthetic CT image and a CT scan image, in accordance with some embodiments.

FIG. 9 is a block diagram of a computer system, in accordance with some embodiments.

### DETAILED DESCRIPTION

This description of the exemplary embodiments is intended to be read in connection with the accompanying drawings, which are to be considered part of the entire written description.

Various embodiments of the present disclosure address the foregoing challenges associated with signal transmission in nuclear imaging, such as positron emission tomography (PET) imaging, for example, by generating a synthetic computerized—tomography (CT) scan for attenuation correction.

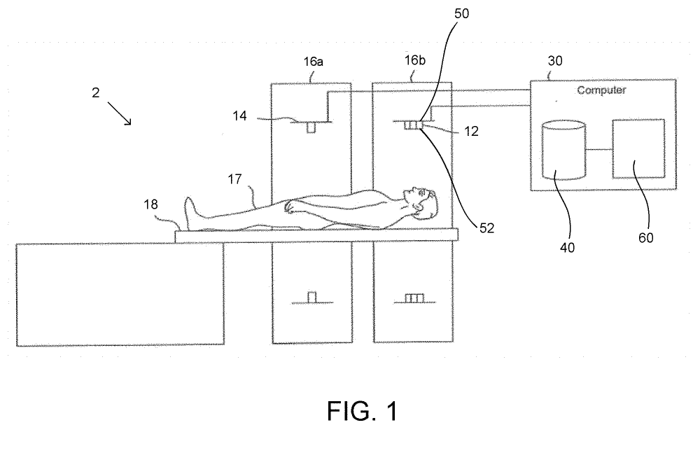

FIG. 1 illustrates one embodiment of a nuclear imaging detector 2. The nuclear imaging detector 2 includes a scanner for at least a first modality 12 provided in a first gantry 16b. The first modality 12 includes a plurality of detectors 50 each having a plurality of crystals 52 configured to detect an annihilation photon, gamma ray, and/or other nuclear imaging event. In various embodiments, the first modality 12 is a PET detector, a single-photon emission tomography (SPECT), and/or any other suitable detector. A patient 17 lies on a movable patient bed 18 that may be movable between a gantry. In some embodiments, the nuclear imaging detector 2 includes a scanner for a second imaging modality 14 provided in a second gantry 16a. The second imaging modality 14 can be any suitable imaging modality, such as, for example, CT, single-photon emission tomography (SPECT) and/or any other suitable imaging modality.

Scan data from the first modality 12 is stored at one or more computer databases 40 and processed by one or more computer processors 60 of a computer 30. The graphical depiction of computer 30 in FIG. 1 is provided by way of illustration only, and computer 30 may include one or more separate computing devices. The imaging data sets can be provided by the first modality 12 and/or may be provided as a separate data set, such as, for example, from a memory coupled to the computer 30. The computer 30 can include one or more processing electronics for processing a signal received from one of the plurality of detectors 50. In some embodiments, and as described in greater detail below, the processor 60 is configured to execute one or more computer executable instructions and perform one or more steps for generating a nuclear image.

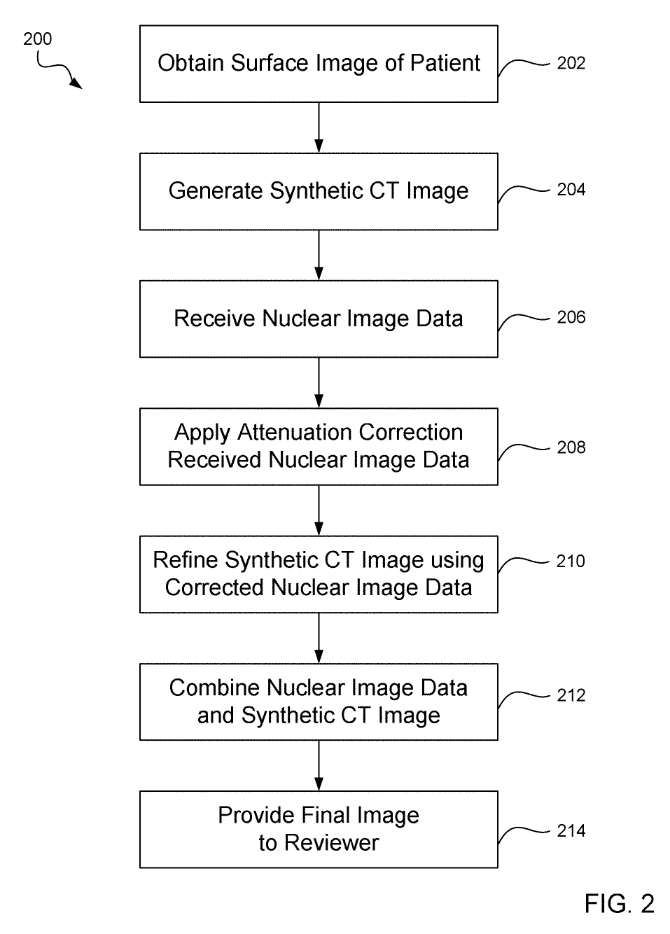

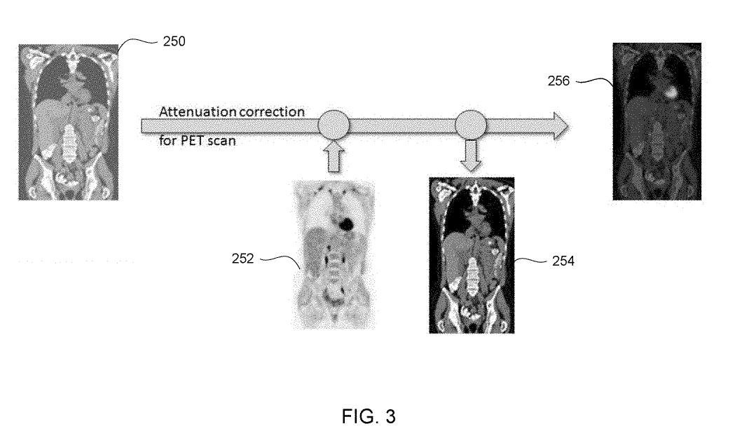

FIG. 2 illustrates a method 200 of generating a combined nuclear image using a synthetic CT scan, in accordance with some embodiments. FIG. 3 illustrates various steps of the method 200 of FIG. 2. With reference now to FIGS. 2 and 3, the method 200 is discussed. At step 202, a surface image of a patient is obtained. The surface image can be generated using any suitable imaging device, such as a three-dimensional (3D) camera. In some embodiments, the second imaging modality 114 is a surface imaging modality.

At step 204, a synthetic CT image 250 is generated from the surface image. The synthetic CT image 250 is a 3D image where each voxel provides an estimate of the expected material density based on a density distribution over a large dataset of patients. The synthetic CT image can illustrate a total volume and/or a partial volume of a patient. The synthetic CT image 250 can have a predicted internal anatomy including, but not limited to, internal body markers (such as a lung center or thyroid), organ surfaces (such as lung, heart, etc.) and/or any other suitable internal anatomy. The synthetic CT image 250 provides measures of attenuation of energy at different locations within the patient, such as one or more voxels. In some embodiments, the synthetic CT image 250 is generated by employing a model based approach to fit a depth image data of a person. The model based approach can include applying one or more learning algorithms including a statistical correlation model between the detailed patient surface geometry (from the surface image) and the anatomical structures (generated from a collection of real and/or synthetic body scans). For example, U.S. Pat. No. 9,524,582, entitled “Method and System for Constructing Personalized Avatars Using a Parameterized Deformable Mesh” discloses methods and systems for generating a synthetic image, and is incorporated by reference herein in its entirety. As shown in FIG. 3, the synthetic CT image 250 provides a surface depth model of the patient. The synthetic CT image 250 is similar to a CT image obtained by a CT imaging modality but does not require doping the patient with additional nuclear imaging isotopes.

In some embodiments, the synthetic CT image 250 is a 3D image similar to a CT volume, where each voxel stores the predicated and/or expected density based on partial measurements such as patient body geometry obtained from a surface image. The estimated image generates sufficient information to control attenuation to generate a homogenous noise value over the entire patient scan area. In some embodiments, the synthetic CT image 250 can be generated based on a formula including a linear combination of skin mask, various organ masks, bone masks, etc., for example:

CT_{synth}=wₛₖᵢₙ*V^{skin}+w_{lungs}*V^{lungs}+wₚₑₗᵥᵢₛ*V^{pelvis}+wₛₖᵤₗₗ*Vₛₖᵤₗₗ . . . (1)

In some embodiments, the weights (w) are set based on the mean density within the specified body regions. The mean density can be based on Hounsfield units (HU); for example, the average density within the lung region is −800 HU and the average density of soft tissue (e.g., skin) is close to o HU.

In some embodiments, a regression method is applied to estimate a surface of internal anatomical structures from the surface image. The regression method can be applied to organs having a much higher and/or lower than average densities, such as, for example, lungs (lower density), pelvis (higher density), shoulders, etc. The organ surfaces and/or internal body markers are determined and a volumetric mask is generated to represent the organ/body marker volume.

In some embodiments, a volumetric regression method is applied to generate organ volume directly from skin surface volume data. In some embodiments, the organ volume can be generated based on a linear regression formula, for example:

A*[Body Surface Volume]=[Volume with Anatomical Structure] (2)

where A is the regression matrix, [Body Surface Volume] is a vector representation of the 3D volume/matrix having voxels inside the body surface marked as 1 and voxels not inside the body surface marked as 0, and [Volume with Anatomical Structure] is a vector representation of the 3D volume/matrix with voxels inside an organ or bone region marked as 1 and voxels outside the organ or bone region marked as 0.

In some embodiments, for a volumetric regression, the individual input and output samples are defined in a fixed space. Variations in organs and body shape for different individuals and therefore a fixed normalized volume must be defined for both input and output. In one embodiment, a fixed 3D volume is considered with a particular body marker as a fixed point (such as body center as volume center or neck center a volume top center). In another embodiment, the volumes may be normalized based on multiple body markers such that in the normalized space, the number of voxels from neck to ischium is fixed across all patients. In some embodiments, a non-linear projection manifold learning technique can be applied to ensure the generated masks are physically plausible.

At step 206, a nuclear image data is received. For example, in some embodiments, a nuclear image 252 is generated by a nuclear imaging modality 112, such as a PET imaging modality, a SPECT imaging modality, and/or any other suitable nuclear imaging modality. The nuclear image data can be generated based on one or more discrete imaging positions and/or based on continuous motion (such as bed motion) imaging. In other embodiments nuclear image data is received by a processor, such as processor 60. In some embodiments, the nuclear image data includes reconstructed time-of-flight (TOF) for detected emissions (such as positron emissions) along one or more lines of response. As used herein, references to nuclear image 252 are equivalent to references to the nuclear image data.

At step 208, attenuation correction is applied to the received nuclear image data. For example, TOF data can be corrected for attenuation, caused by, for example, one or more organs, bones, and/or other structures in the patient. In some embodiments, the synthetic CT image 250 can be used as a density map for attenuation correction during step 206. The synthetic CT image 250 indicates organs, masses, and/or other elements that may absorb photons during a nuclear imaging process. By combining the synthetic CT image 250 with the PET scan data, an attenuation corrected PET image 252 can be generated. Any suitable method of attenuation correction can be applied. For example, in various embodiments, MLAA (maximum likelihood attenuation map activity), MLACF (maximum likelihood attenuation correction factors), and/or any other suitable method can be applied. In some embodiments, as discussed below, non-corrected PET image can be used.

At step 210, the synthetic CT image 250 is refined based on the nuclear image 252 to generate a refined CT image 254. The refined CT image 254 includes additional detail and/or error correction as compared to the synthetic CT image 250. In some embodiments, patient skin surface boundaries, boundaries of anatomical structures, body markers (such as a lung center), etc. can be identified in the nuclear image 252. The identified boundaries and/or body markers can be applied to the synthetic CT image 250 to further refine the boundary and/or body marker locations of the CT image 250. For example, in some embodiments, a space carving method is applied to deform the initial synthetic CT image 250 to fit the PET measurements and re-project the measurements back on the manifold of valid volume masks generated for the synthetic CT scan 250. In some embodiments, the refined the synthetic CT image 250 is not refined.

At step 212, a combined nuclear image 256 is generated by combining the nuclear image 252 and the refined CT image 254 (or the synthetic CT image 250), according to one or more known methods. For example, in some embodiments, the combined nuclear image 256 is generated by overlaying voxels of the nuclear image 252 with corresponding voxels of the refined CT image 254, although it will be appreciated that any suitable method of combining a nuclear image 252 and the refined CT image 254 can be used.

At step 214, the combined nuclear image 256 is provided to a reviewer. The reviewer can be any suitable person trained to read fusion images, such as the combined nuclear image 256. In some embodiments, the final image 256 can be provided to the reviewer on the computer 30 coupled to the nuclear imaging detector 2, a remote system in signal communication with the computer 30, and/or any other suitable review modality.

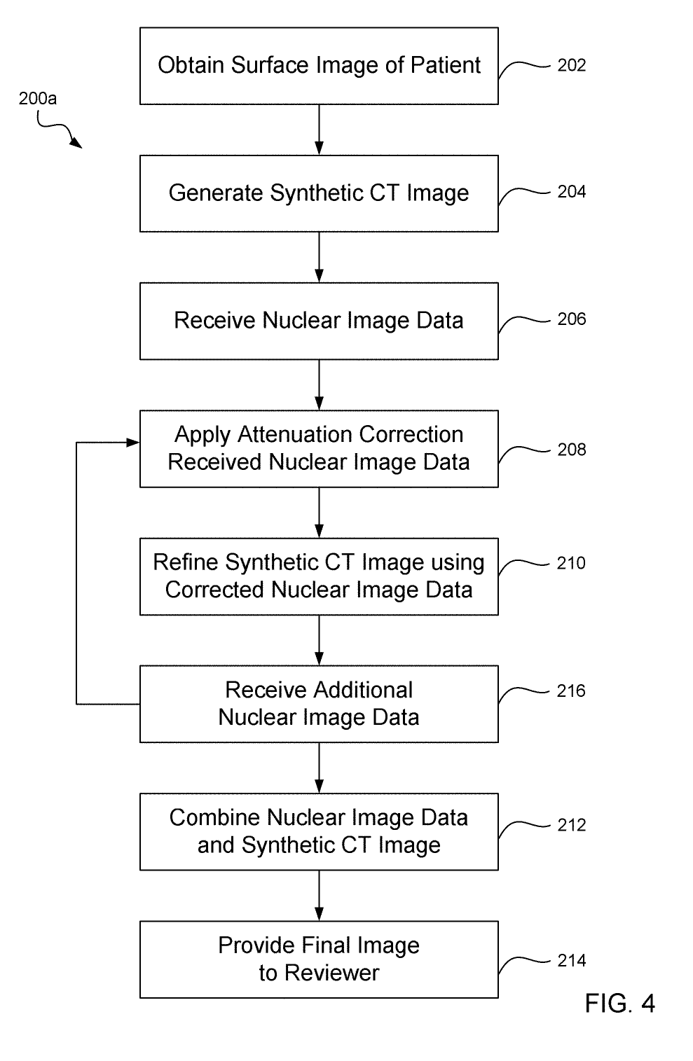

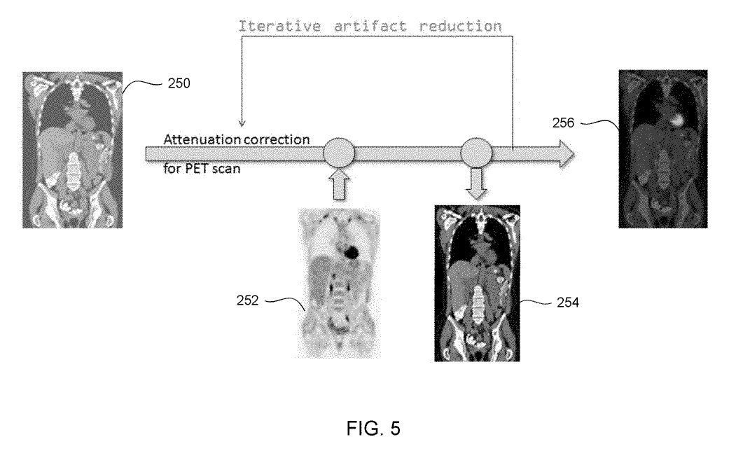

FIG. 4 illustrates a method 200a of generating a combined nuclear image using a synthetic CT 250 scan including an iterative artifact reduction process, in accordance with some embodiments. FIG. 5 illustrates various steps of the method 200a, in accordance with some embodiments. The method 200a is similar to the method 200 discussed above, and similar description is not repeated herein. At discussed above, in some embodiments, the synthetic CT image 250 is applied as a density map for attenuation correction during a nuclear imaging process. The synthetic CT image 250 identifies density differences throughout the body to allow for correction of photon absorption by the body during a nuclear imaging process. The synthetic CT image 256 can be refined based on the nuclear image 252, as discussed above with respect to step 210. In some embodiments, the refined CT image 254 can be used as a density map for additional nuclear imaging scans in an iterative process.

For example, at step 216, additional nuclear image data is obtained. The method 200a repeats step 208 to apply attenuation correction to the additional nuclear image data using the refined CT image 254 as a density map for attenuation correction. The refined CT image 254 provides improved attenuation correction over the original synthetic CT image 250, as the refined CT image 254 has improved accuracy of anatomical structures and/or boundaries. The use of the refined CT image 254 provides an additional nuclear image, such as PET image, having greater precision than the nuclear image 252 obtained using the synthetic CT image 250.

After obtaining an additional nuclear image and applying attenuation correction (using the refined synthetic CT image 254), the method 200a repeats steps 210 to further refine the CT image 254 based on the additional nuclear image. The increase in accuracy of the additional nuclear image (as compared to a prior nuclear image 252) allows the patient skin surface boundaries, boundaries of anatomical structures, body markers (such as a lung center), etc. of the refined CT image 254 to be adjusted. The method 200a can iteratively repeat steps 216 and 210 to generate additional nuclear images and apply further refinements to the CT image 254. After a predetermined number of iterations, the method 200a continues to step 212 and proceeds as discussed above with respect to method 200.

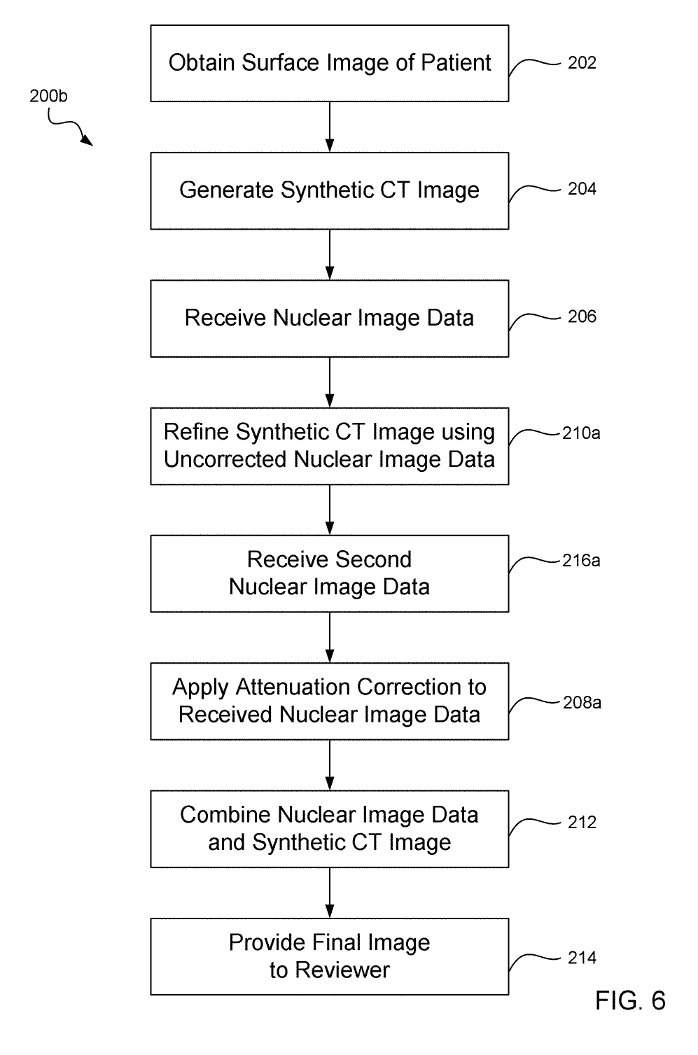

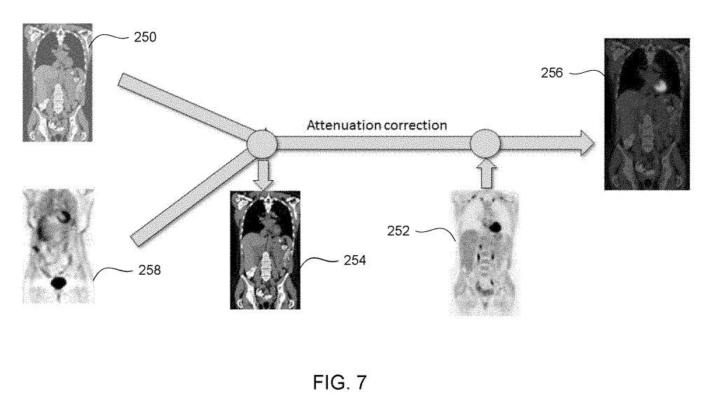

FIG. 6 illustrates a method 200b of generating a combined nuclear image 256 using an initial uncorrected nuclear image, in accordance with some embodiments. FIG. 7 illustrates various steps of the method 200b, in accordance with some embodiments. The method 200b is similar to the method 200 discussed above, and the description for steps 202 through 206 is not repeated herein. At step 210a, the synthetic CT image 250 is refined based on an uncorrected nuclear image 258 to generate the refined CT image 254, as discussed above.

At step 216a, second nuclear image data is obtained. At step 208a, attenuation correction is applied to the second nuclear image data using the refined CT image 254 as a density map. The additional nuclear image data has increased accuracy and fewer errors due to the application of attenuation correction. The additional nuclear image data can be combined with the refined CT image 254 to generate a combined nuclear image 256 as discussed above at step 212.

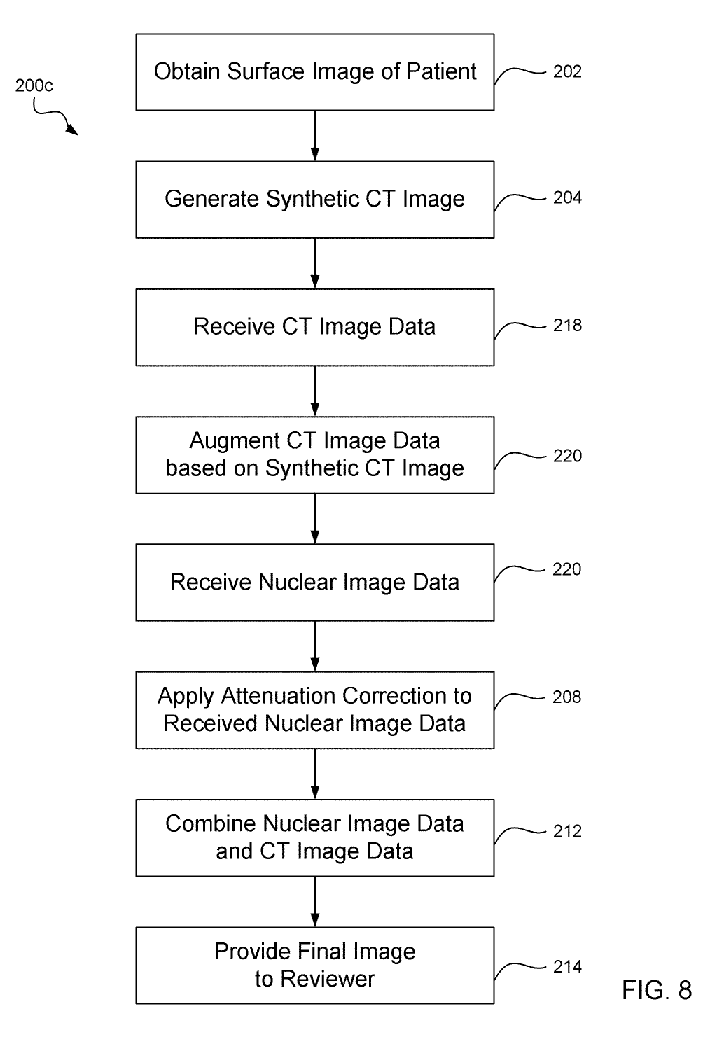

FIG. 8 illustrates a method 200c of generating a fusion image 256 using a synthetic CT image 250 in conjunction with a CT scan image obtained during a CT scan, in accordance with some embodiments. The method 200c is similar to the method 200 discussed above, and the description for steps 202 and 204 is not repeated herein. At step 218, CT scan image data is received. The CT scan image data can be obtained by an imaging modality, such as a second imaging modality 114. In other embodiments, the CT scan image data is received from a remote system. The CT scan image can be obtained prior to, simultaneously with, and/or after obtaining a nuclear image 252 at step 206. In some embodiments, the CT scan image data includes truncated image data in some areas as compared to the synthetic CT scan 250. As used herein, references to a CT scan image are equivalent to references to the CT scan image data.

At step 220, the CT scan image is augmented and/or modified by applying the synthetic CT scan 250 to complete truncated regions of the CT scan image and to provide input for scatter background events estimation. The augmented CT image includes CT scan data obtained during the CT scan (at step 216) and synthetic CT image data (generated from the surface depth data). For example, in some embodiments, the CT scan image 262 is supplemented using surface depth data generated from the surface image without generating a complete synthetic CT image 250.

At step 206, a nuclear image is obtained. At step 208, attenuation correction is applied to the nuclear image data using the augmented CT image as a density map. For example, in some embodiments, an initial nuclear image 252 is obtained at step 206 after obtaining the augmented CT image. The augmented CT image is used in place of the synthetic CT image 250 for attenuation correction. As another example, in some embodiments, an additional nuclear image 258 is additional nuclear image data. The additional nuclear image can be generated from TOF data obtained prior to, during, and/or after obtaining an initial nuclear image 252 and/or performing a CT scan. U.S. Pat. No. 9,155,514, entitled “Reconstruction with Partially Known Attenuation Information in Time of Flight Positron Emission Tomography,” issued Oct. 13, 2015, discloses additional information regarding PET attenuation correction using CT image data and is incorporated by reference herein in its entirety.

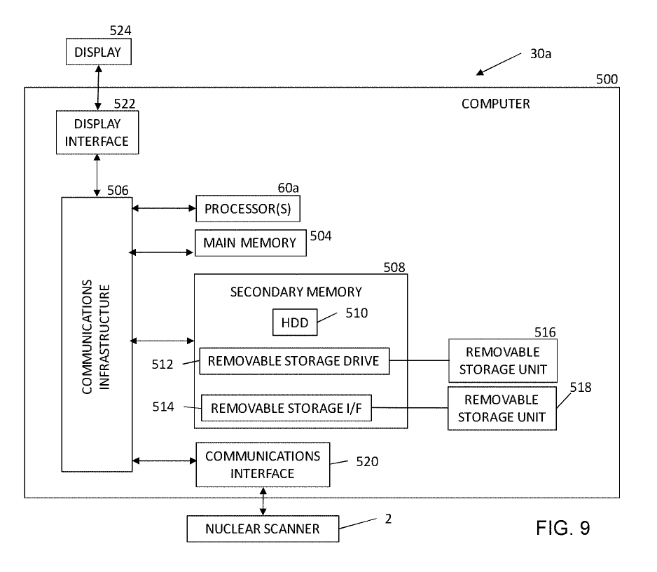

FIG. 9 is a block diagram of a system 500 for generating a nuclear image. The system 500 includes the nuclear imaging detector 2 and a computer system 30a. The computer system 30a can be used in some embodiments, e.g., for implementing the system 30 controlling the nuclear imaging detector 2. Computer system 30a may include one or more processors 60a. Each processor 60a is connected to a communication infrastructure 506 (e.g., a communications bus, cross-over bar, or network). The processor 60a can be implemented as a central processing unit, an embedded processor or microcontroller, an application-specific integrated circuit (ASIC), and/or any other circuit configured to execute computer executable instructions to perform one or more steps. Processors 60a are similar to the processor 60 discussed above and similar description is not repeated herein. Computer system 30a may include a display interface 522 that forwards graphics, text, and other data from the communication infrastructure 506 (or from a frame buffer, not shown) for display on the display unit 524 to a user.

Computer system 30a may also include a main memory 504, such as a random access memory (RAM), and a secondary memory 508. The main memory 504 and/or the secondary memory 508 comprise a dynamic random access memory (DRAM). The secondary memory 508 may include, for example, a hard disk drive (HDD) 510 and/or removable storage drive 512, which may represent a solid state memory, an optical disk drive, a flash drive, a magnetic tape drive, or the like. The removable storage drive 512 reads from and/or writes to a removable storage unit 516. Removable storage unit 516 may be an optical disk, magnetic disk, floppy disk, magnetic tape, or the like. The removable storage unit 516 may include a computer readable storage medium having tangibly stored therein (or embodied thereon) data and/or computer executable software instructions, e.g., for causing the processor(s) to perform various operations and/or one or more steps.

In alternative embodiments, secondary memory 508 may include other devices for allowing computer programs or other instructions to be loaded into computer system 30a. Secondary memory 508 may include a removable storage unit 518 and a corresponding removable storage interface 514, which may be similar to removable storage drive 512, with its own removable storage unit 516. Examples of such removable storage units include, but are not limited to, universal serial bus (USB) or flash drives, which allow software and data to be transferred from the removable storage unit 516, 518 to computer system 30a.

Computer system 30a may also include a communications interface (e.g., networking interface) 520. Communications interface 520 allows instructions and data to be transferred between computer system 30a and nuclear imaging detector 2. Communications interface 520 also provides communications with other external devices. Examples of communications interface 520 may include a modem, Ethernet interface, wireless network interface (e.g., radio frequency, IEEE 802.11 interface, Bluetooth interface, or the like), a Personal Computer Memory Card International Association (PCMCIA) slot and card, or the like. Instructions and data transferred via communications interface 520 may be in the form of signals, which may be electronic, electromagnetic, optical, or the like that are capable of being received by communications interface 520. These signals may be provided to communications interface 520 via a communications path (e.g., channel), which may be implemented using wire, cable, fiber optics, a telephone line, a cellular link, a radio frequency (RF) link and other communication channels.

The methods and system described herein may be at least partially embodied in the form of computer-implemented processes and apparatus for practicing those processes. The disclosed methods may also be at least partially embodied in the form of tangible, non-transitory machine-readable storage media encoded with computer executable program code. The media may include, for example, RAMs, ROMs, CD-ROMs, DVD-ROMs, BD-ROMs, hard disk drives, flash memories, or any other non-transitory machine-readable storage medium, wherein, when the computer program code is loaded into and executed by a computer, the computer becomes an apparatus for practicing the method. The methods may also be at least partially embodied in the form of a computer into which computer program code is loaded and/or executed, such that, the computer becomes a special purpose computer for practicing the methods. When implemented on a general-purpose processor, the computer program code segments configure the processor to create specific connections, circuits, and algorithms for implementing the methods disclosed herein.

The apparatuses and processes are not limited to the specific embodiments described herein. In addition, components of each apparatus and each process can be practiced independent and separate from other components and processes described herein.

The previous description of embodiments is provided to enable any person skilled in the art to practice the disclosure. The various modifications to these embodiments will be readily apparent to those skilled in the art, and the generic principles defined herein may be applied to other embodiments without the use of inventive faculty. The present disclosure is not intended to be limited to the embodiments shown herein, but is to be accorded the widest scope consistent with the principles and novel features disclosed herein.

What is claimed is:

1. A method for generating a nuclear image, comprising:
obtaining, via a camera, a surface image of a patient;
generating a synthetic computed-tomography (CT) image of the patient based on the surface image;
obtaining, via a nuclear imaging modality, first time-of-flight (TOF) data of the patient;
applying attenuation correction to the first TOF data, wherein the synthetic CT image is applied as a density map during the attenuation correction; and
generating a nuclear image from the attenuation corrected first TOF data.

2. The method of claim 1, comprising refining the synthetic CT image based on the attenuation corrected first TOF data.

3. The method of claim 2, comprising generating an output image by overlaying the nuclear image with the refined synthetic CT image.

4. The method of claim 1, further comprising:
obtaining, via the nuclear imaging modality, second TOF data of the patient; and
applying attenuation correction to the second TOF data, wherein the refined synthetic CT image is applied as a density map during the attenuation correction.

5. The method of claim 1, further comprising, prior to obtaining the first TOF data:
obtaining, via the nuclear imaging modality, non-corrected TOF data from the patient; and
refining the synthetic CT image of the patient based on the non-corrected TOF data.

6. The method of claim 1, wherein the synthetic CT image is generated by a model based approach.

7. The method of claim 6, wherein the model based approach includes at least one of a statistical correlation model, a linear combination, or a volumetric regression.

8. The method of claim 1, wherein the attenuation correction includes at least one of MLAA (maximum likelihood attenuation map activity) or MLACF (maximum likelihood attenuation correction factors).

9. The method of claim 1, further comprising:
obtaining, via a CT imaging modality, a CT scan image of the patient; and
augmenting the CT scan image based on the synthetic CT image to complete truncated regions of the CT scan image.

10. The method of claim 9, comprising:
obtaining second TOF data of the patient; and
applying attenuation correction to the second TOF data, wherein the augmented CT scan image is applied as a density map during attenuation correction.

11. The method of claim 1, wherein the nuclear imaging modality is one of a positron-emission tomography (PET) modality or a single-photon emission computerized tomography (SPECT) modality.

12. A system for generating a nuclear image, comprising:
a gantry sized and configured to receive a patient;
a scanner including a first imaging modality configured to detect a first plurality of photon events, the first imaging modality comprising a plurality of detectors;
a camera configured to obtain a surface image of the patient; and
a processor configured to receive a signal indicative of the first plurality of photon events and the surface image, wherein the processor is configured to:
generate a synthetic computed-tomography (CT) image of the patient based on the surface image;
apply attenuation correction to the first plurality of photon events, wherein the synthetic image is applied as a density map during the attenuation correction; and
generate a nuclear image from the attenuation corrected plurality of photon events.

13. The system of claim 12, wherein the processor is further configured to refine the synthetic CT image based on the attenuation corrected first TOF data.

14. The system of claim 13, wherein the first imaging modality is configured to detect a second plurality of photon events, wherein the processor is configured to apply attenuation correction to the second plurality of photon events, and wherein the refined synthetic CT image is applied as a density map during the attenuation correction.

15. The system of claim 12, wherein the processor is configured to generate a non-corrected nuclear image from the first plurality of photon events prior to applying attenuation correction, and wherein the processor is configured to refine the synthetic CT image based on the non-corrected nuclear image.

16. The system of claim 12, wherein the synthetic CT image is generated by a model based approach.

17. The system of claim 12, further comprising:
a CT imaging modality configured to obtain partial attenuation information for the patient, wherein the processor is configured to augment the partial attenuation information based on the synthetic CT image to complete truncated regions of the partial attenuation information, and wherein the attenuation correction of the nuclear image is based on the augmented attenuation information.

18. A non-transitory computer-readable medium encoded with computer executable instructions, the computer executable instructions, when executed by a computer in a system for generating a nuclear image, cause the system for generating a nuclear image to execute the steps of:
obtaining a surface image of a patient;
generating a synthetic computed-tomography (CT) image of the patient based on the surface image;
obtaining first time-of-flight (TOF) data of the patient;
applying attenuation correction to the first TOF data, wherein the synthetic CT image is applied as a density map during the attenuation correction; and
generating a nuclear image from the attenuation corrected first TOF data.

19. The non-transitory computer-readable of claim 18, wherein the computer executable instructions cause the computer to further execute the steps of:
refining the synthetic CT image based on the attenuation corrected first TOF data. obtaining second TOF data;
applying attenuation correction to the second TOF data, wherein the refined synthetic CT image is applied as a density map during the attenuation correction; and
generating an output image by overlaying the second TOF data with the refined synthetic CT image.

20. The non-transitory computer-readable of claim 18, wherein the computer executable instructions cause the computer to further execute the steps of:
obtaining partial attenuation information;
augmenting the partial attenuation information based on the synthetic CT image to complete truncated regions of the partial attenuation information; and
applying attenuation correction to the first TOF data, wherein the augmented attenuation information is applied as a density map during the attenuation correction.
